(19) United States
(12) Patent Application Publication
(10) Pub. No.: US 2022/0168335 A1
(43) Pub. Date: Jun. 2, 2022

(54) Hyaluronic Acid Derivatives for Wound Healing

(71) Applicants: Queen's University at Kingston, Kingston (CA); Jilin University, Changchun City, Jilin (CN)

(72) Inventors: Jianfeng Guo, Jilin (CN); Yin Gao, Jilin (CN); Tassos Anastassiades, Kingston (CA)

(21) Appl. No.: 17/437,067

(22) PCT Filed: Mar. 6, 2020
(86) PCT No.: PCT/CA2020/000022

§ 371 Date: Sep. 8, 2021

Related U.S. Application Data
(60) Provisional application No. 62/815,782, filed on Mar. 8, 2019.

(51) Int. Cl. A61K 31/728; A61P 17/02; A61K 9/00; A61K 47/36

(52) U.S. Cl. CPC A61K 31/728; A61K 47/36; A61K 9/0014; A61P 17/02

(57) ABSTRACT

A composition and method for promoting wound healing that includes hyaluronic acid derivatives, and in particular, derivatives in which the N-acetyl group of hyaluronic acid has been substituted.

### FIELD

The present disclosure relates to hyaluronic acid derivatives, and in particular, derivatives in which the N-acetyl group of hyaluronic acid has been substituted, and methods and uses thereof.

### INTRODUCTION

The integrity of healthy skin can protect body from external harm, sense environmental changes, and maintain physiological homeostasis. Therefore, skin regeneration and repair after surgical wounds, acute trauma and chronic diseases (e.g. diabetes) are a central concern of healthcare Although wound healing generally proceeds efficiently after the onset of a lesion, poor outcome may follow larger injuries or a variety of pathological states, such as infection and vascular disease. impaired cutaneous wound healing may become life-threatening and is a major public health issue worldwide.

The process of wound healing is highly organized, including three primary phases: inflammation, proliferation, and maturation. Recently, an improved understanding of the cellular and molecular mechanisms underlying these phases has advanced the development of novel regenerative and reparative therapies (Pang, C., et al., (2017) Int. Wound J. 14(3): 450.459). These approaches include administration of growth factors (Barrientos, S., et al., (2014) Wound Repair Regan. 22(5):569-78), cell reprogramming (Teng, M. et al., (2014) Wound Repair Regan. 22(2)151-60), and tissue engineering (Sun, B. K. et al., (2014) Science 346(6212) 941-5) and have demonstrated potential in the protection and renewal of the skin. Hurdles remain for the application of these approaches to cutaneous lesions; for example, the administration of growth factors lacks appropriate drug delivery systems, and the efficacy of cell-based strategies may be dampened by the complexities including the pathological conditions of donors, onset time and duration of treatment, and dose and route of administration. Therefore, viable and efficient alternatives are still needed.

Hyaluronic acid (HA) is a glycosaminoglycan (GAG) of the extracellular matrix (ECM), which plays important roles in the adhesion, proliferation and differentiation of cells during embryogenesis (embryonic development), morphogenesis, and tissue regeneration. These biological activities differ greatly depending on the molecular weight of HA. For example, high molecular weight HA (HMW-HA) possesses anti-inflammatory or immunosuppressive activities, while low molecular weight HA (LMW-HA) demonstrates pro-inflammatory or immunostimulatory behaviors. In addition, HMW-HA displays anti-angiogenic properties, whereas LMW-HA is able to promote the formation of new blood vessels.

### SUMMARY

In one embodiment, the invention provides a composition for promoting wound healing in a subject, comprising a pharmaceutically acceptable excipient or carrier, and a therapeutically effective amount of a hyaluronic acid derivative comprising repeating units of a disaccharide of Formula (I), wherein a portion of the disaccharide units of Formula(I) have been independently replaced with a disaccharide structure of Formula (II) wherein R is —C(O)—(C₂-C₂₀)-alkyl, —C(O)—(C₂-C₂₀)-alkenyl or —C(O)—(C₂-C₂₀)-alkynyl, or a pharmaceutically acceptable sodium- or potassium-salt, ester, or glucoside thereof.

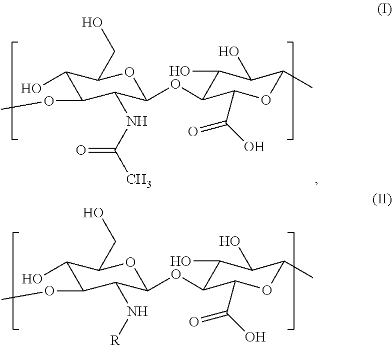

wherein the hyaluronic acid derivative has a molecular weight of at least about 20 kDa, and wherein the hyaluronic acid derivative promotes wound healing.

In one embodiment, the wound is a topical wound. In one embodiment, the wound comprises a cut, abrasion, diabetic wound, canker, ulcer, aphthous stomachitis, sore, burn, surgical wound, abrasion, and/or surgical adhesion. A wound can be caused by tissue damage due to, for example, chemical damage, thermal damage, bites, trauma, etc. In one embodiment, the wound is a chronic wound. In one embodiment, the wound is an acute wound.

In one embodiment, the invention provides a method for promoting wound healing in a subject, the method comprising: administering to a subject a formulation that comprises a hyaluronic acid comprising repeating units of a disaccharide comprising glucuronic acid and N-acetylglucosamine, wherein a portion of the N-acetyl groups of the N-acetylglucosamine have been independently replaced with a group of the formula —N—C(O)—(C₂-C₂₀)-alkyl, —N—C(O)—(C₂-C₂₀)-alkenyl or —N—C(O)—(C₂-C₂₀)-alkynyl, and wherein the hyaluronic acid derivative has a molecular weight of at least about 20 kDa, or a pharmaceutically acceptable sodium or potassium salt, ester, or glucoside thereof.

In one embodiment, the formulation is a topical formulation. In one embodiment, the administering comprises applying to the skin. In one embodiment, the administering comprises oral administration. In one embodiment, the wound is a chronic wound. In one embodiment, the wound is an acute wound. In one embodiment, the wound comprises a diabetic wound, canker, ulcer, aphthous stomachitis, sore, burn, surgical wound, abrasion, and/or surgical adhesion.

In one aspect the invention provides a composition for promoting topical wound healing in skin of a subject, comprising a pharmaceutically acceptable excipient or carrier, and a therapeutically effective amount of a hyaluronic acid derivative comprising repeating units of a disaccharide of Formula (I), wherein a portion of the disaccharide units of Formula(I) have been independently replaced with a disaccharide structure of Formula (II) wherein R is —C(O)—(C₂-C₂₀)-alkyl, —C(O)—(C₂-C₂₀)-alkenyl or —C(O)—(C₂-C₂₀)-alkynyl , or a pharmaceutically acceptable sodium- or potassium-salt, ester, or glucoside thereof.

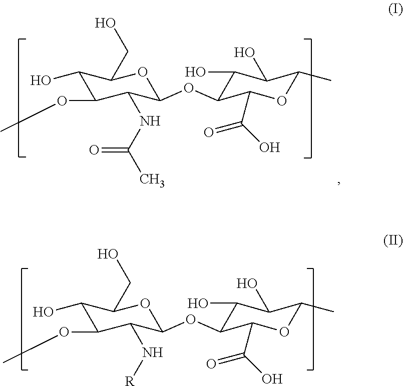

wherein the hyaluronic acid derivative has a molecular weight of at least about 20 kDa, and wherein the hyaluronic acid derivative promotes wound healing.

In one embodiment, the hyaluronic acid derivative is cross-linked. In one embodiment, R is —C(O)—(C₂₋₄) alkyl. In one embodiment, the portion of N-acetyl groups which are replaced is at least about 10%. In one embodiment, the portion of N-acetyl groups which are replaced is between about 20% to about 80%. In one embodiment, the molecular weight is at least about 30 kDa. In one embodiment, the molecular weight is between about 20 kDa to about 250 kDa. In one embodiment, the amount of hyaluronic acid derivative is about 0.05 to about 1 mg/mL, In one embodiment, the amount of hyaluronic acid derivative is 0.25 mg/mL. In one embodiment, the composition further comprises moisturizer, emollient, thickener, preservative, and/or a firming agent. In one embodiment, the composition further comprises sodium carboxymethylcellulose, or 1-(3-dimethylaminopropyl)-3-ethyl-carbodiimide hydrochloride. In one embodiment, the moisturizer is glycerol, petroleum jelly, petrolatum, propylene glycol, butylene glycol, Lactic acid or a salt thereof, erythriol, D-panthenol, PEG-n, or 2-methacryloyloxyethyl phosphorylcholine. In one embodiment, wherein the emollient is ceramides, cholesterol, fatty acids, mineral oil, polyalkylene, methyl glucoside, squalene, or a combination thereof. In one embodiment, the humectant is hyaluronic acid, glycerin, sorbitol, N-acetylethanolamine, glycereth, sodium pyroglutamate, urea, or a combination thereof. In one embodiment, the thickener is sodium alginate, an anionic polysaccharide, alginic acid, alginates, pectin, carrageenan, xanthan gum, carboxy methyl cellulose, a cationic polysaccharide, chitosan, polyguanternium-4, polyquanternium-10, a non-ionic polysaccharide, guar gum, hydroxypropyl guar, locust bean gum, sclerotium, methyl cellulose, hydroxyethyl cellulose, carbopol, acrylate copolymer, mineral salt, magnesium aluminium silicate, and/or a surface active agent, potassium stearate, betaine. In one embodiment, the preservative is ethyl hydroxybenzoate, hydroxyphenyl or an ester or salt thereof, methylparaben, benzylparaben, sodium methylparaben, sodium butylparaben, isothiazolinone, methyltchloroisothiazolinone, methylisothiazolinone, acidic preservative, benzoic acid, sorbic acid, alcohol, bronopol, phenoxyethanol, quaternary ammonium salt, benzalkonium chloride, benzethonium chloride, aldehyde, (benzyloxy)methanol, glutaric dialdehyde, phenol, chlorophene, chloroxylenol, or a combination thereof. In one embodiment, the firming agent is calcium gluconate, calcium chloride,

In one aspect the invention provides a method for promoting topical wound healing in skin of a subject, the method comprising: applying to the skin of a subject a topical formulation that comprises a hyaluronic acid comprising repeating units of a disaccharide comprising glucuronic acid and N-acetylglucosamine, wherein a portion of the N-acetyl groups of the N-acetylglucosamine have been independently replaced with a group of the formula —N—C(O)—(C₂-C₂₀)-alkyl, —N—C(O)—(C₂-C₂₀)-alkenyl or —N—C(O)—(C₂-C₂₀)-alkynyl, and wherein the hyaluronic acid derivative has a molecular weight of at least about 20 kDa, or a pharmaceutically acceptable sodium or potassium salt, ester, or glucoside thereof.

In one embodiment, the wound is a chronic wound. In one embodiment, the wound is an acute wound. In one embodiment, the wound comprises a diabetic wound, canker, ulcer, aphthous stomachitis, sore, burn, surgical wound, bite, abrasion, surgical adhesion, and/or tissue damage due to chemical damage, thermal damage, or trauma.

### DRAWINGS

For a better understanding of the invention and to show more clearly how it may be carried into effect, reference will be made by way of example to the accompanying drawings, which illustrate aspects and features according to embodiments of the invention, and in which:

FIG. 1 shows a plot of healing rate (%) of untreated control group, Blank-Gel, CMC and BHA-Gel on days 3, 5, 7, 10 and 14 when compared with the wound area on Day 0 (n=6), # p<0.05 and ## p<0.01 relative to untreated control group; & p<0.05 and && p<0.01 relative to Blank-Gel: * p<0.05 and ** p<0.01 relative to CMC.

FIG. 2A-C shows a plot of protein level of (A) TNF-α, (B) IL6 and (C) IL1β in wounds from rats (n=6) treated with Blank-Gel, CMC, or BHA-Gel as determined by ELISA and shown as fold change to those of rats in untreated control group. ## p<0.01 relative to untreated control group; && p<0.01 relative to Blank-Gel; * p<0.05 and ** p<0.01 relative to CMC.

FIG. 3A-C shows a plot of (A) the relative expression of phosphorated TAK-1 protein, (B) the nuclear p65 protein level and (C) the relative expression of phosphorated p38 protein in wounds of rats (n=6) treated with Blank-Gel, CMC, BHA-Gel was determined using western blotting on Day 3, 5, 7, 10 and 14. ## p<0.01 relative to untreated control group; && p<0.01 relative to Blank-Gel; * p<0.05 and ** p<0.01 relative to CMC.

FIG. 4 shows a plot of TGF-β1 protein level in wounds of rats (n=6) treated with Blank-Gel, CMC, BHA-Gel was determined using western blotting and was shown as the fold change in each sample relative to those of rats without treatment. ## p<0.01 relative to untreated control group; && p<0.01 relative to Blank-Gel; ** p<0.01 relative to CMC.

FIGS. 5A-C show mRNA level of (A) Smad2, (B) Smad3 and (C) Smad7 in wounds of rats (n=6) treated with Blank-Gel, CMC, BHA-Gel was determined by RT-PCR and was shown as the fold change to those of rats in untreated control group. # p<0.05 and ## p<0.01 relative to untreated control group; & p<0.05 and && p<0.01 relative to Blank-Gel; * p<0.05 and ** p<0.01 relative to CMC.

FIG. 6A-D show mRNA level of (A) VEGF, (B) eNOS, (C) E-selectin and (D) Integrin-β3 in the wound of rats (n=6) treated with Blank-Gel, CMC, BHA-Gel was determined by RT-PCR and was shown as the fold change to those of rats in untreated control group. # p<0.05 and ## p<0.01 relative to untreated control group; & p<0.05 and && p<0.01 relative to Blank-Gel; * p<0.05 ** p<0.01 relative to CMC.

FIG. 7A-B shows protein level of (A) collagen III and (B) collagen I in wounds of rats (n=6) treated with Blank-Gel, CMC, or BHA-Gel as determined using western blotting and shown as fold change in each sample relative to those of rats without treatment. ## p<0.01 relative to untreated control group; && p<0.01 relative to Blank-Gel; ** p<0.01 relative to CMC.

### DESCRIPTION

### Definitions

The term “hyaluronic acid” or “hyaluronan” is known in the art and as used herein refers to the glycosaminoglycan polymer composed of repeating units of the disaccharide comprised of glucoronic acid, for example D-glucoronic acid, and N-acetylglucosamine, for example, D-N-acetylglucosamine.

The term “derivative” as used herein refers to a substance which comprises the same basic carbon skeleton and functionality as the parent compound, but can also bear one or more substituents or substitutions of the parent compound. The term “derivative” includes those chemical modifications which involve the replacement of a portion of the N-acetyl groups of the N-acetylglucosamine of hyaluronic acid with a different acyl group or with a hydrogen. The term derivative also includes compounds in which a portion of the N-acetyl groups are reacetylated. Other derivatives include, for example, ester derivatives and include any compounds in which, in one embodiment, free hydroxyl groups of hyaluronic acid have been esterified (e.g. methyl esters, ethyl esters, benzyl esters etc.).

The term “cross-linked” as used herein means that two or more hyaluronic acid derivatives are covalently bonded inter-molecularly through a suitable cross-linking compound or agent or cross-linker. Alternatively, the cross-linking occurs intra-molecularly between sites of the same hyaluronic acid derivative. The cross-linking compound reacts with free hydroxyl groups, free carboxyl groups and/or free amino groups of the hyaluronic acid derivatives to form the cross-linked hyaluronic acid derivatives.

The term “cross-linker” or “cross-linking compound” or “cross-linking agent” as used herein refers to a compound which can react with at least two free hydroxyl groups, free carboxyl groups and/or free amino groups on a hyaluronic acid derivative as described in the present disclosure, and then react a second time to form a cross-linked hyaluronic acid derivative. The cross-linker can react intermolecularly to cross-Ink two or more different hyaluronic acid derivatives or intra-molecularly to cross-link two different positions of the same hyaluronic acid derivative.

The term “substituted” or “replaced” as used herein means that the N-acetyl group of the N-acetylglucosamine of hyaluronic acid is replaced with a selection from the indicated groups.

The term “a portion” as used herein refers to a part or fraction of the N-acetyl groups of the N-acetylglucosamine being substituted with a different acyl group or a hydrogen atom, or bonded to a cross-linker. For example, between 1 and 100% of the N-acetyl groups are replaced.

The term “N-acetyl group” as used herein is known in the art and refers the N-acetyl functionality of N-acetylglucosamine and has the chemical formula —N—C(O)—CH₃.

The term “alkyl” as used herein refers to straight or branched chain, saturated alkyl groups. The term (C₂-Cₙ)-alkyl means an alkyl group having at least two carbon atoms, and up to “n” carbon atoms, depending on the identity of “n”. For example, (C₂-C₂₀)-alkyl includes alkyl groups having 2, 3, 4, 5, 6, 7, 8, 9, 10, 11, 12, 13, 14, 15, 16, 17, 18, 19 or 20 carbon atoms, and includes ethyl, propyl, isopropyl, butyl, sec-butyl, iso-butyl, etc.

The term “alkenyl” as used herein, whether it is used alone or as part of another group, means straight or branched chain, unsaturated alkenyl groups. The term (C₂-Cₙ)-alkenyl means an alkenyl group having at least two carbon atoms, and up to “n” carbon atoms, depending on the identity of “n”. For example, (C₂-C₂₀-alkenyl includes alkenyl groups having 2, 3, 4, 5, 6, 7, 8, 9, 10, 11, 12, 13, 14, 15, 16, 17, 18, 19 or 20 carbon atoms, and includes ethenyl, propenyl, isopropenyl, butenyl, sec-butenyl, iso-butenyl, etc.

The term “alkynyl” as used herein, whether it is used alone or as part of another group, means straight or branched chain, unsaturated alkynyl groups. The term (C₂-Cₙ)-alkynyl means an alkynyl group having at least two carbon atoms, and up to “n” carbon atoms, depending on the identity of “n”. For example, (C₂-C₂₀)-alkynyl includes alkynyl groups having 2, 3, 4, 5, 6, 7, 8, 9. 10, 11, 12, 13, 14, 15, 16, 17, 18, 19 or 20 carbon atoms, and includes ethynyl, propynyl, isopropynyl, butynyl, sec-butynyl, iso-butynyl, etc.

The term “pharmaceutically acceptable salt” refers, for example, to a salt that retains the desired biological activity of a compound of the present disclosure and does not impart undesired toxicological effects thereto; and may refer to an acid addition salt or a base addition salt.

The term “acid addition salt” as used herein means any non-toxic organic or inorganic salt of any basic compound. Basic compounds that form an acid addition salt include, for example, compounds comprising an amine. For example, an acid addition salt includes any non-toxic organic or inorganic salt of any basic compound of the present disclosure. Inorganic acids that may form suitable salts include, without limitation, hydrochloric, hydrobromic, sulfuric and phosphoric acids, as well as metal salts such as sodium monohydrogen orthophosphate and potassium hydrogen sulfate. Organic acids that may form suitable salts include, without limitation, mono-, di-, or tricarboxylic acids such as glycolic, lactic, pyruvic, rnalonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono- or di-acid salts may be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to a person skilled in the art.

The term “base addition salt” as used herein means any non-toxic organic or inorganic base addition salt of any acidic compound. Acidic compounds that form a base addition salt include, for example, compounds comprising a carboxylic acid group, For example, a base addition salt includes any non-toxic organic or inorganic base addition salt of any acidic compound of the present disclosure. Inorganic bases that may form suitable salts include, without limitation, lithium, sodium, potassium, calcium, magnesium or barium hydroxide. Organic bases that may form suitable salts include, without limitation, aliphatic, alicyclic or aromatic organic amines such as methylamine, trimethylamine and picoline or ammonia. The selection of the appropriate salt will be known to a person skilled in the art. Those of ordinary skill in the art will recognize further pharmaceutically acceptable salts for the compounds provided herein. In general, a pharmaceutically acceptable acid addition salt or base addition salt is synthesized from a parent compound that contains a basic or acidic moiety by any conventional chemical method. For example, a neutral compound is treated with an acid or a base in a suitable solvent and the formed salt is isolated by filtration, extraction or any other suitable method.

In embodiments of the present disclosure, the compounds described herein have at least one asymmetric center. These compounds exist as enantiomers. Where compounds possess more than one asymmetric center, they may exist as diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (e.g. less than 20%, suitably less than 10%, more suitably less than 5%) of compounds of the disclosure having alternate stereochemistry. For example, compounds of the disclosure that are shown without any stereochemical designations are understood to be racemic mixtures (i.e. contain an equal amount of each possible enantiomer or diastereomer). However, it is to be understood that all enantiomers and diastereomers are included within the scope of the present disclosure, including mixtures thereof in any proportion.

As used herein, the terms “treating” or “treatment” and the like refer to an approach for obtaining beneficial or desired results, including clinical results. Beneficial or desired clinical results may include, without limitation, alleviation or amelioration of one or more symptoms or conditions, diminishment of extent of disease, stabilization (i.e. not worsening) of the state of disease, prevention of development of disease, prevention of spread of disease, delay or slowing of disease progression, delay or slowing of disease onset or progression, amelioration or palliation of the disease state, diminishment of the reoccurrence of disease and remission (whether partial or total), whether detectable or undetectable. “Treating” or “treatment” may also refer to prolonging survival of a subject as compared to that expected in the absence of treatment. “Treating” or “treatment” may also refer to inhibiting the progression of disease, slowing the progression of disease temporarily or halting the progression of the disease permanently.

The term “administered” as used herein means administration of a therapeutically effective dose of a compound or composition of the disclosure to a subject.

The term “effective amount” or “therapeutically effective amount” as used herein means an amount effective, at dosages and for periods of time necessary to achieve the desired result. For example, in the context of treating a subject with cancer, an effective amount is an amount that, for example, reduces the tumor volume compared to the tumor volume without administration of the compound of the present disclosure. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art.

As used herein, a “subject” refers to all members of the animal kingdom including mammals, and suitably refers to humans. A member of the animal kingdom includes, without limitation, a mammal (such as a human, primate, swine, sheep, cow, equine, horse, camel, canine, dog, feline, cat, tiger, leopard, civet, mink; stone marten, ferret, house pet, livestock, rabbit, mouse, rat, guinea pig or other rodent, seal, whale and the like), fish, amphibian, reptile, and bird (such as water fowl, migratory bird, quail, duck, goose, poultry, or chicken). in an embodiment of the present disclosure, the subject is in need of a compound or composition of the disclosure.

As used herein, the term “wound” refers to a chronic or acute injury to living tissue which typically means the tissue is damaged, cut, or broken. Wounds include for example, but not limited to, cuts, diabetic wounds, cankers, ulcers, aphthous stomachitis, sores, burns, abrasions, surgical wounds, and/or surgical adhesions.

### Embodiments

Hyaluronan (hyaluronic acid) is a widely distributed glycosaminoglycan in animal tissues, composed of alternating monosaccharide units of N-acetyl glucosamine (N-acetyl-2-amide glucose) and glucuronic acid. Hyaluronan has multiple functions including hydration, provision of matrix for cell migration and lubrication of joints. intact hyaluronan has a high molecular mass of greater than 1,000 kDa but can exist in lower molecular mass forms, for example, 100-250 kDa. Intact hyaluronan is often derived commercially from rooster comb or from bacterial sources. High molecular mass hyaluronans have high viscosity, which is important in lubricant properties of joints. However, the size and likely folding of the greater than 1,000 kDa hyaluronans presents a different physico-chemical milieu to cell receptors and the organization of interacting matrix macromolecules, than the smaller molecular mass forms. The high molecular mass hyaluronan is believed to be degraded enzymatically to lower mass fragments in tissues. A range of hyaluronic acid (HA) fragments using low molecular weight hyaluronans (LMW-HA) have been produced. An exemplary LMW-HA was prepared, which was a polymer modified with N-butyrylated moieties (BHA).

As described in Singh et al. 2017 (Surgery, 35:9 473-477), regardless of aetiology of a wound, all tissue repair processes are similar. A wound results in a coordinated physiological response in the tissue to begin processes of inflammation, proliferation and remodeling regardless of the nature, location, or tissue type of the wound in a subject. Thus, wound healing in accordance with the compositions and methods described herein applies to healing of any damaged tissue, wherein the damage is chronic or acute injury, such as, but not limited to, trauma, cuts, diabetic wounds, cankers, ulcers, aphthous stomachitis, sores, burns, abrasions, surgical wounds, and/or surgical adhesions.

Results described herein demonstrate that BHA has demonstrated potential for dermal wound healing in vitro and in vivo relative to a control. The control was a commercial wound care product that included carboxymethyl chitosan. These results are compared to the “parent” partially de-acetylated LMW-HA (“DHA”) and re-acetylated DHA (“AHA”). DHA and AHA delayed dermal wound repair relative to other control groups. This result demonstrates the critical role of acylation of LMW-HA in wound repair.

BHA-mediated skin repair was investigated by targeting three phases of wound healing: 1) the inflammatory phase was modulated via downregulation of NF-κB and MAPK signal pathways; 2) the proliferative phase was enhanced due to the promotion of epithelialization, angiogenesis and lymphangiogenesis; 3) the maturation phase was facilitated by remodeling of collagens from type III to type I. These results demonstrated significant potential of BHA for clinical translation in cutaneous wound healing.

The present disclosure relates to hyaluronic acid derivatives in which the N-acetyl group of hyaluronic acid has been removed or substituted with a different acyl functionality. Accordingly, in one embodiment, there is included a hyaluronic acid derivative comprising repeating units of a disaccharide unit comprising D-glucuronic acid and D-N-acetylglucosamine moieties, wherein a portion of the N-acetyl groups of the D-N-acetylglucosamine of the disaccharide unit have been substituted or replaced with hydrogen or a group of the formula —N—C(O)—(C₂-C₂₀)-alkyl, —N—C(O)—(C₂-C₂₀)-alkenyl or —N—C(O)—(C₂-C₂₀)-alkynyl, wherein the hyaluronic acid derivative has a molecular weight of at least about 20 kDa, or a pharmaceutically acceptable salt, ester, or glucoside thereof.

In one embodiment, the disaccharide repeating unit of the hyaluronic acid derivative has the structure of the Formula (I), wherein a portion of the disaccharide units of the Formula (I) in the hyaluronic acid derivative are substituted or replaced with disaccharide units of the Formula (II), wherein R is H, —C(O)—(C₂-C₂₀)-alkyl, —C(O)—(C₂-C₂₀)-alkenyl or —C(O)—(C₂-C₂₀)-alkynyl.

In one embodiment, R is H, —C(O)—(C₂-C₁₆)-alkyl, —C(O)—(C₂-C₁₆)-alkenyl or —C(O)—(C₂-C₁₆)-alkynyl. In another embodiment, R is H, —C(O)—(C₂-C₁₀)-alkyl, —C(O)—(C₂-C₁₀)-alkenyl or —C(O)—(C₂-C₁₀)-alkynyl. In a further embodiment, R is H, —C(O)—(C₂-C₆)-alkyl, —C(O)—(C₂-C₆)-alkenyl or —C(O)—(C₂-C₆)-alkynyl. In an embodiment, R is H or —C(O)—(C₂-C₅)-alkyl. In another embodiment, R is H, -—C(O)-propyl, —C(O)-butyl, —C(O)-pentyl, —C(O)-isopentyl, or —C(O)-hexyl.

In other embodiments of the disclosure, the portion of N-acetyl groups (or portion of disaccharide units of Formula (I) replaced with units of Formula (II)) which are substituted is at least about 10%, or at least about 20%. In another embodiment, the portion of N-acetyl groups which are substituted is between about 10% - 100%, or optionally between about 20% to about 80%. In other embodiments, the portion of N-acetyl groups which are substituted is at least about 1%, 2%, 5%. 10%, 20%, 30%. 40%, 50%, 60%, 70%, 80%, 90%, 95%, 98%, 99%, or 100%.

In another embodiment, the hyaluronic acid derivative of the present disclosure generally has a lower molecular weight when compared with the parent hyaluronic acid. Hyaluronic acid generally has a molecular weight of at least about 1,000 kDa. In one embodiment, the hyaluronic acid derivatives of the disclosure have a molecular weight of at least about 25 kDa, or at least about 30 kDa. In other embodiments, the derivative has a molecular weight of between about 20 kDa to about 500 kDa, or between about 20 kDa to about 250 kDa, or between about 50 kDa to about 250 kDa.

In another embodiment of the disclosure, the hyaluronic acid derivatives also include derivatives which have been re-acetylated. Accordingly, in one embodiment, there is included a hyaluronic acid derivative comprising repeating units of a disaccharide comprising D-glucuronic acid and D-N-acetylglucosamine, wherein a portion of the N-acetyl groups of the D-N-acetylglucosamine have been substituted with an N-acetyl functionality, and wherein the hyaluronic acid derivative has a molecular weight of at least about 20 kDa, or a pharmaceutically acceptable salt, ester, or glucoside thereof.

In one embodiment, free hydroxyl groups, free carboxyl groups and/or free amine groups (when R is H) in the hyaluronic acid derivatives of the present disclosure, are reacted with a suitable cross-linker to form cross-linked hyaluronic acid derivatives, in which one or more hyaluronic acid derivatives of the present disclosure are cross-linked to form cross-linked polymers having varying degrees of gelation.

In another embodiment, when R is H, a portion of the disaccharide units of the Formula (II), are replaced with disaccharide units of the Formula (III), in which the free amine group (—NH₂) are reacted with a suitable cross-linker to form the structure of Formula (III), wherein X is any suitable cross-linker.

In one embodiment, the cross-linker is biocompatible. in another embodiment, the cross-linker is divinyl sulfone (DVS), 1-ethyl-3-(3-dimethylaminopropyl) (glutaraldehyde), 1-ethyl-3-(3-dimethylaminopropyl)carbodiimide (EDC), poly(ethyelene glycol) diglycidyl ether (EX 810), 3.3¢-dithiobis(propanoic dihydrazide) (DTPH), 1,3,5-benzene(tricarboxylic trihydrazide), or poly(ethylene glycol)-diamine tetrapropanoic tetrahydrazide. In another embodiment, the cross-linker is (1R,4aS,5,7aS-tetrahydro-1-hydroxy-7-(hydroxymethyl)-cyclopenta[c]pyran-4carboxylic acid, methyl ester (genipin)). Cross-linking reactions occur between, for example, the said free amines of the hyaluronic acid derivative (when R is H) and the cross-linker (such as genipin) to yield hyaluronic derivative polymers with various degrees of gelation. Such polymers may be admixed or further cross-linked with connective tissue components such as collagens and other matrix proteins and glycoproteins to form gels of different composition and biomechanical properties.

In another embodiment, the hyaluronic acid derivatives of the present disclosure are cross-linked to biological, synthetic or biodegradable polymers, such as chitosan or collagen. In another embodiment, the hyaluronic acid derivatives of the present disclosure are cross-linked to glycoproteins, proteoglycans, proteins, peptides, or poly(2-hydroxyethylmethacrylate).

In one embodiment, the hyaluronic acid derivatives are in a mixture with HMW HA in a ratio of 1:99 up to 99:1 of hyaluronic acid derivative:HMW HA.

In one embodiment, the hyaluronic acid derivative comprises repeating units of a disaccharide which has the structure of the Formula (I), wherein a portion of the disaccharide units of the Formula (I) in the hyaluronic acid derivative are substituted or replaced with disaccharide units of the Formula (II, wherein R is H, —C(O)—(C₂-C₂₀)-alkyl, —C(O)—(C₂-C₂₀)-alkenyl or —C(O)—(C₂-C₂₀)-alkynyl, and wherein a portion of the disaccharide units of the Formula (II) are substituted or replaced with disaccharide units of the Formula (III), wherein X is any suitable cross-linker.

In one embodiment, the hyaluronic acid derivative comprises repeating units of a disaccharide which has the structure of the Formula (I), wherein a portion of the disaccharide units of the Formula (I) in the hyaluronic acid derivative are substituted or replaced with disaccharide units of the Formula (H), wherein R is H, R is H, —C(O)—(C₂-C₂₀)-alkyl, —C(O)—(C₂-C₂₀)-alkenyl or —C(O)—(C₂-C₂₀)-alkynyl, and wherein a portion of the disaccharide units of the Formula (II are substituted or replaced with disaccharide units of the Formula (III), wherein X is any suitable cross-linker, in which the disaccharide units of the Formula (III) cross-link to form a structure of the Formula (IV).

In one embodiment, the cross-linked hyaluronic acid derivative has the structure of the Formula (IV), wherein

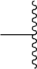

indicates the repeating disaccharide units of the Formula (I),

(II) and (III) of the hyaluronic acid derivatives, wherein at least a portion units of the Formula (III) are cross-linked to another hyaluronic acid derivative to form cross-linked structures of the Formula (IV).

In one embodiment, the invention provides a composition for wound healing. One representative example of this composition is known herein as BHA -gel. See Table 1 for details regarding the formulation of BHA-gel. Once component of the formulation is a moisturizer (e.g., Glycerol, petroleum jelly, petrolatu ucoside, or squalene). One component is a humectant, which has hydrophilic and/or viscous moieties that attract water (e.g., hyaluronic acid, which may be a variety of molecular weight ranges and may or may not include BHA and should not be confused with an active agent that is described herein, glycerin, sorbitol, N-acetylethanolamine, glycereth, sodium pyroglutamate and urea). Another component is a thickener such as, for example, sodium m, 2-acetamidoethanol, Lactic acid, PEG-n that includes hydrophobic and/or viscous components that form a barrier over skin to prevent water from escaping. Another component is an emollient (e.g., ceramides, cholesterol, fatty acids, mineral oil, polyalkylene, methyl gl alginate, an anionic polysaccharide (e.g., alginic acid and alginates, pectin, carrageenan, xanthan gum, carboxy methyl cellulose), a cationic polysaccharide (e.g., chitosan, polyquanternium-4 and 10), a non-ionic polysaccharide (e.g., guar gum, hydroxypropyl guar, locust bean gum, sclerotium, methyl cellulose, hydroxyethyl cellulose), carbopol, acrylate copolymer, mineral salt (e.g., magnesium aluminium silicate), and/or a surface active agent (e.g., Potassium stearate, Betaine). Another component is a preservative such as, for example, ethyl hydroxybenzoate. Other examples of preservatives include hydroxyphenyl or an ester or salt thereof (e.g., methylparaben, benzylparaben, sodium methylparaben, sodium butylparaben), isothiazolinones (e.g., methyltchloroisothiazolinone, methylisothiazolinone), Acidic preservative (e.g., benzoic acid, sorbic acid), Alcohols (e.g., Bronopol, Phenoxyethanol), Quaternary Ammonium Salt (e.g., Benzalkonium chloride, Benzethonium chloride), Aldehyde (e.g., (benzyloxy)methanol, Glutaric dialdehyde), or Phenol (e.g., Chlorophene, Chloroxylenol). Another component is a firming agent or sequestrant, and/or texturizer (e.g., calcium gluconate, calcium chloride) as a source of divalent ions for cross-linking of the thickener (e.g., sodium alginate).

TABLE 1
Formulation of BHA-gel

| Component (use) | Formula |
|---|---|
| Glycerol (moisturizer) | 450 |
| Alginic acid sodium salt (thickener) | 30 |
| Ethyl 4-hydroxybenzoate (preservative) | 2.00 |
| Calcium gluconate (increases the gel | 0.50 |
| viscosity) | |
| BHA | 0.25 |
| Ultrapure water | 517.25 |
| | 1000 g |

As described in the examples provided herein, an exemplary LMW-HA (i.e., BHA) was investigated to see if it would modulate inflammatory stage and facilitate proliferation and remodeling stages of wound healing. A synthetic BHA (˜39 kDa) containing 0% NH₂, 73.3±3.2% N-acetyl, 28.6±0.6% N-butyryl moieties was investigated for its healing efficacy in rats with excisional full-thickness wounds. Specifically, a gel formulation (BHA-Gel, see Table 1) was applied to locally release BHA into wounds. It is noteworthy that the therapeutic effect of BHA-Gel was dependent on the administration dose. The healing efficacy was improved when the dose was increased from 0.05 to 0.25 mg/mL. However, the skin repair was less effective at the doses of 0.5 and 1 mg/mL. Therefore, the BHA-Gel with a dose of 0.25 mg/mL was used for in vivo experiments.

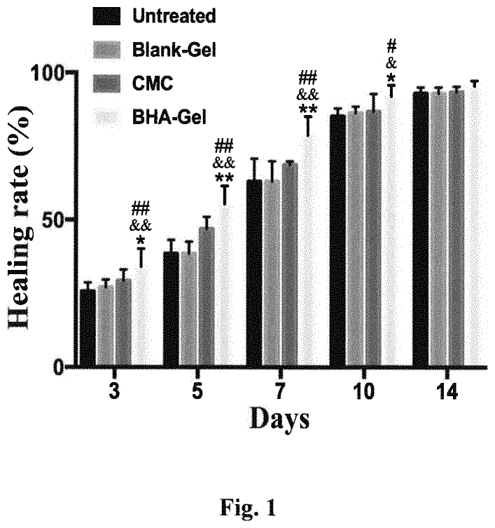

Referring to FIG. 1, BHA-Gel significantly promoted dermal wound repair on Day 3 to 10 when compared to an untreated control group and a Blank-Gel. Healing efficacy was also significantly improved by BHA-Gel relative to the carboxymethyl chitosan (CMC, the active ingredient of a commercial wound care product, CHITIN®). In addition, in comparison with the other control groups, the skin layer was more organised following the treatment of BHA-Gel, demonstrating the development of discrete skin structures including increased blood vessels and hair follicles. Also, there was reduced infiltration of pro-inflammatory cells and epidermal hyperplasia. In addition, no significant difference was observed between untreated control group and Blank-Gel, indicating that the healing effect of the BHA-Gel resulted from BHA (see FIG. 1), confirming that BHA effectively promotes cutaneous wound healing, including the development of discrete skin structures.

In contrast to BHA, two of the resultant acylated HA fragments, namely AHA (˜45 kDa, containing 0.0% NH₂, 97.7±1.4% N-acetyl, and 0.0% N-butyryl moieties) and DHA (˜42 kDa, containing 21.6±1.1% NH₂, 78.4±0.6% N-acetyl, and 0.0% N-butyryl moieties) significantly delayed the dermal wound repair relative to other control groups. These results further confirm that BHA promotes skin wound healing when the naturally-occurring N-acetyl group of HA is replaced with the longer N-acyl chain of the N-butyryl group. These results demonstrate the critical role and specificity of N-acylation of LMW-HA in wound healing.

During the early stages of wound healing (˜1 to 3 days after injury), neutrophils and macrophages as the dominant pro-inflammatory cells, regulate local and systemic defense responses to the wound. However, increased pro-inflammatory cells may prolong the inflammatory response and delay the healing process, thus causing nonhealing (chronic) wounds. Consequently, elevated levels of pro-inflammatory cytokines are often observed in chronic wounds.

Toll-like receptor 4 (TLR4), a transmembrane protein belonging to the toil-like receptor family, regulates both innate and adaptive immune responses. It has been reported that TLR4 is highly activated during the early stages of cutaneous wound healing and regulates pro-inflammatory cytokine production at the sites of injury. The stimulation of TLR4 by lipopolysaccharide (LPS, a major component of Gram-negative bacteria) can induce the activation of the nuclear factor-κB (NF-κB and mitogen-activated protein kinases (MAPK) signaling pathways, leading to the induction and release of pro-inflammatory cytokines.

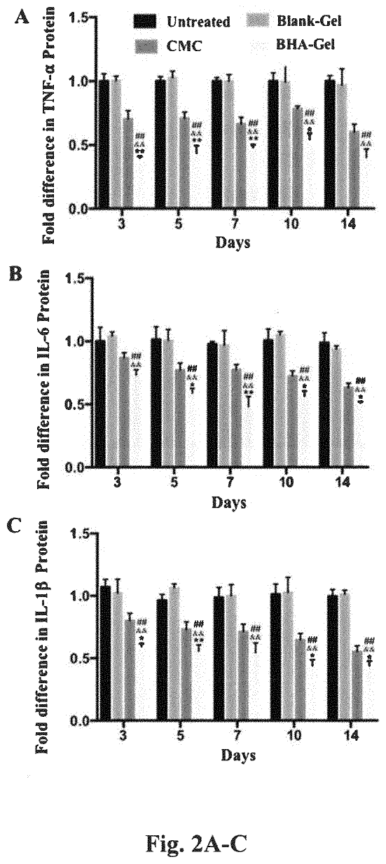

BHA can significantly attenuate the cytokine production [e.g. tumor necrosis factor-α (TNF-α), interleukin 6 (IL,6) and IL-1β] stimulated by LPS through the TLR-4 in vitro (Babasola, O., et al., J. Biol. Chem. 289(36) (2014) 24779-91). Therefore, the anti-inflammatory effects of BHA in the same manner were further confirmed in vivo. As shown in FIG. 2A-C, the TNF-α, IL-6 and IL-1β protein levels in skin samples were determined using ELISA, indicating that BHA-Gel significantly reduced the production of these pro-inflammatory cytokines in wounds relative to the other control groups,

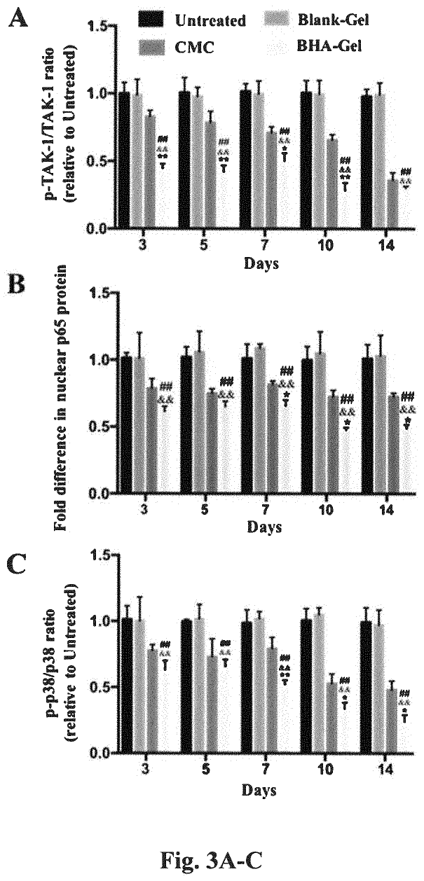

Mitogen-activated protein kinase, kinase 7 (MAP3K7, also known as TGF-β activated kinase 1, TAK-1) acts as a key mediator in TLR4-mediated signaling pathways, and the phosphorylation of TAK-1 results in TAK-1-dependent activation of NF-κB and MAPK signaling pathways. Therefore, the expression of TAK-1 protein (including the phosphorylated form) in skin samples was evaluated using western blotting (see FIG. 3A), indicating that BHA-Gel significantly inhibited the expression of phosphorylated TAK-1 protein. As a result, the expression of nuclear-p65 protein (p65, a gene product from the NF-κB transcription factor complex; the transportation of p65 protein into nucleus activates the NF-κB pathway) was also significantly reduced accordingly (FIG. 3B). In addition, the activation of p38 MAP kinase, an important member of the MAPK kinase family, was also significantly suppressed by BHA-Gel (FIG. 3C). These results confirmed that BHA could reduce the pro-inflammatory cytokine production in vivo by suppressing TLR-4-mediated NF-κB and MAPK signal cascades, and likely promoting cutaneous wound healing during the inflammation phase.

During a proliferative phase, ˜3 to 10 days after injury, along with the recovery of wound surface (re-epithelialization), there is restoration of the vascular network (angiogenesis and lymphangiogenesis). Fibroblasts are one of the most abundant cell types in injured sites, and play a key role in the re-epithelialization process during wound healing. CD44 (a transmembrane glycoprotein widely found on diverse cell types, e.g. fibroblasts in the skin) regulates cell-cell and cell-matrix interactions during dermal wound repair. Recent studies have demonstrated the role of CD44 in the adhesion and motility of fibroblasts for tissue repair (Acharya, P. S., et al., J. Cell Sci. 121 (Pt 9) (2008) 1393-402). For example, fibroblast migration can be mediated by CD44-dependent pathways (Acharya, P. S., et al., J. Cell Sci. 121 (Pt 9) (2008) 1393-402), and the downregulation of CD44 in mice with excisional injury caused impaired fibrotic activities during the early stages of wound healing (Govindaraju, P., et al., Matrix. Biol. 75-76 (2019) 314-30).

The interplays between intact HA and its principal receptor, CD44, are known to positively regulate the fibroblast activities (Misra, S., et al., Front. Immunol. 6 (2015) 201). Expression of CD44 was significantly upregulated with the treatment of BHA-Gel on Day 3 to 14 relative to the other control groups. These results imply that BHA enhances the activities of dermal fibroblasts in the proliferation phase mostly due to the up-regulation of CD44.

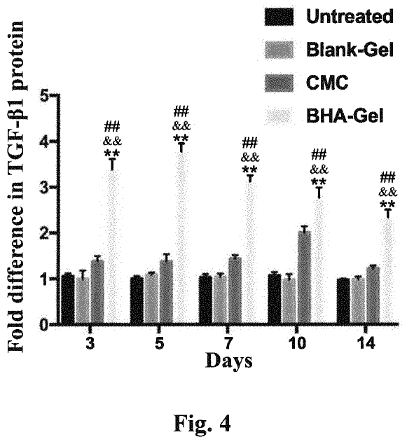

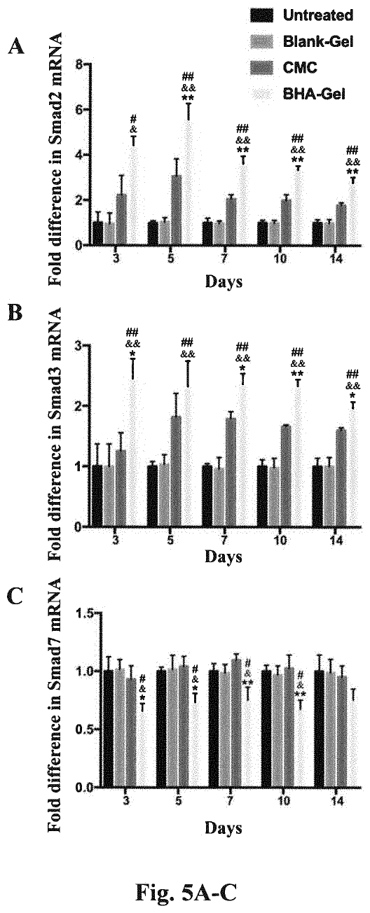

The production of collagen by fibroblasts, as one prerequisite for the new connective tissue matrix (Raja, K., et al., Front. Biosci. 12 (2007) 2849-68), is promoted with the stimulation of potent growth factors (e.g. Transforming growth factor beta 1, TGF-β1) released from macrophages (Koh, T. J. et al., Expert. Rev. Mal. Med. 13 (2011) e23). As shown in FIG. 4), the expression of TGF-β1 protein in skin samples was significantly improved by BHA-Gel relative to the other control groups. It has been reported that TGF-β1 promotes the synthesis and accumulation of ECM proteins by activating the Smad signalling pathway (Choi, M. E., et al., Semin. Nephrol. 32 (3) (2012) 244-52). When the TGF-β1/SMAD-dependent pathway is activated, the downstream targets of TGF-β1 (such as Smad2 and Smad 3) are upregulated (Massague, J., et al., Cell, 103 (2000) 295-309). Indeed, the BHA-Gel also significantly increased the expression of Smad2 and Smad3 (see FIG. 5A and 5B), whereas the expression of Smad 7, a member of Smad family that deactivates Smad2 and Smad3, was significantly downregulated accordingly (FIG. 5C). As a result, the collagen deposition, when analysed using the Masson-Trichrome stain assay, was significantly elevated in dermal samples from the BHA-Gel group relative to other control groups. These confirmed the ability of BHA in the synthesis of new collagen matrix and the resurfacing of wound during the re-epithelialization process.

In addition, the vascular system is recovered during the proliferative phase in order to rebuilt the micro-circulation, increase the oxygen supply, and restore the nutritive perfusion in injured areas. LMW-HA is known as a critical regulator of vascular endothelial cell function. Therefore, the proliferation and migration of endothelial cells mediated by BHA were first assessed in vitro using human umbilical vein endothelial cells (HUVEC). BHA was able to significantly promote the proliferation of HUVEC relative to untreated control group. In addition, the migration of HUVEC was also significantly promoted with the treatment of BHA.

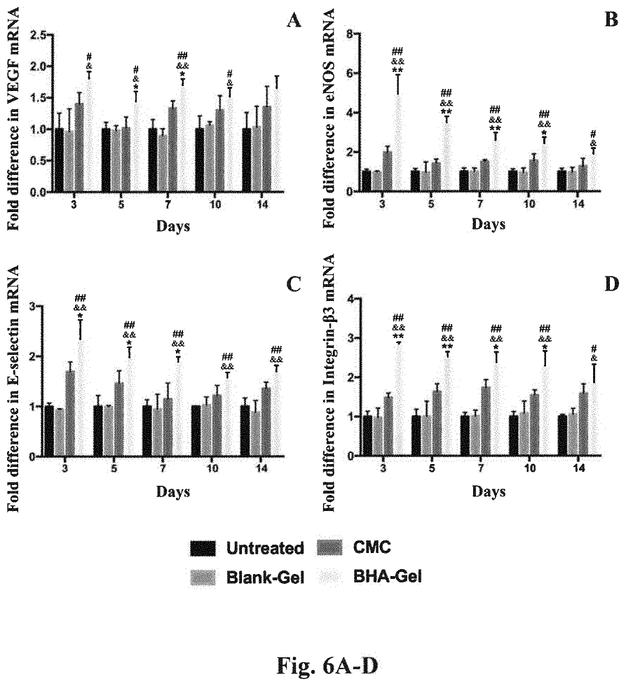

Angiogenic activity of BHA was further assessed in vivo (FIG. 6A-D). It is known that vascular endothelial growth factor (VEGF) regulates the early events (i.e. the endothelial cell proliferation and migration) during the angiogenesis (Barrientos, S., Wound. Repair. Regen. 16(5) (2008) 585-601)). As shown in FIG. 6A, BHA-Gei significantly enhanced the VEGF gene expression in wounds relative to other control groups on Day 3 to 10. In addition, a group of adhesion molecules that are required for increasing endothelial cell proliferation and migration in wound repair, including endothelial nitric oxide synthase (eNOS) (FIG. 6B), E-selectin (FIG. 6C) and integrin-62 3 (FIG. 6D), were also significantly upregulated by BHA-Gel. In addition, the expression of CD31 (a marker for neovascularization (Newman P. J., J. Clin. invest. 99(1) (1997) 3-8)) was also assessed in dermal samples using immunohistochemical staining assay. These results indicate that BHA-gel significantly promoted the expression of CD31 when compared to other control groups, further confirming the role of BHA in facilitating the angiogenesis during wound healing.

Moreover, the lymphatic endothelium formation (lymphangiogenesis) was also examined in vivo in terms of lymph vessel endothelial hyaluronan receptor-1 (LYVE-1, a marker for lymphatic vessels (Jackson D. G., Trends. Immunol. 22(6) (2001) 317-21)) expression. A greater level of new lymphatic vessels were observed in the wound of rats treated with BHA-Gel relative to other control groups on Day 3 to 14, indicating that BHA was able not only to promote angiogenesis but also to enhance the formation of lymphatic vessels in wound healing.

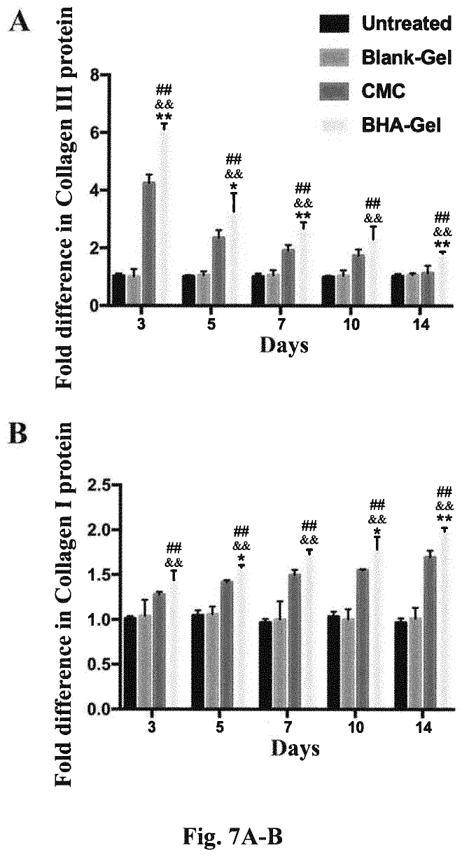

In the later proliferation phase, the granulation tissue is formed on the wound surface via the interplays between fibroblasts, inflammation cells, and epithelial cells (Reinke, J. M., et al., et. al., Eur. Surg. Res. 49(1) (2012) 35-43). The granulation tissue in turn creates a framework for these cells and regulates the proliferation, differentiation, and migration of these cells within it (Eckes, B., et al., Fibrogenesis Tissue Repair 3 (2010) 4) (Barker, T. H., Biomaterials, 32(18) (2011) 4211-4). The favorable behaviors achieved by BHA, including modulation of inflammatory responses (FIG. 2A-C), development of new vascular systems (FIG. 6A-D), and formation of extracellular collagens (FIG. 7A-B), imply that the formation of granulation tissues can lead to the efficient wound closure (see FIG. 1).

ECM remodeling, which is the final step of wound healing, starts after the formation of granulation tissue and results in the reorganization of connective tissue. During this stage, most of endothelial cells and macrophages undergo programmed cell death (apoptosis) and as a result, the wound is left with few cells but a mass of EMC proteins. Collagens are known as the most abundant ECM proteins, and have a diversity of functions in skin including the maintenance of tissue structure and integrity, contribution of tensility, flexibility and softness, and stabilization of epidermal-dermal interface.

Type III collagen, which is mainly produced in the proliferation phase, plays a key role in fibrillogenesis (the development of collagen fibrils in connective tissue) and in regulating collagen fibril diameter. In addition, type I collagen, which is the most abundant collagen in the skin, is known to enhance the skin structure and integrity during the maturation phase. Results in FIG. 7A-B) show that BHA-Gel was able to significantly promote the expression of type III and type I collagens in wounds relative to other control groups.

One hallmark in ECM remodeling is the switch from collagen type III to type I, which is accomplished by the interactions between fibroblasts, macrophages, and endothelial cells. Following treatment of BHA-Gel, the level of type III collagen expression in the wound was slowly decreased from Day 3 to Day 14 (FIG. 7A), whereas the level of type I collagen expression was gradually increased under the same conditions (see FIG. 7B). These results suggest that BHA may promote the collagen remodeling by regulating the balance in the ratio between collagen type Hi and type III.

Collagen remodeling normally ends up with the formation of scar tissues (hypertrophic scar or keloid) in adult human skin. The newly formed scars cannot restore the flexibility or strength of the original skin. it is known that delayed wound repair is strongly associated with scarring (Tracy, L. E., et al., Adv. Wound. Care. (New Rochelle) 5(3)(2016) 119-136), thus requiring therapeutic strategies for accelerating the wound healing and reducing the scar formation. BHA, due to the promise for accelerated wound healing, demonstrated less epidermal hyperplasia when compared to other control groups, in addition, scarless healing has been observed in the fetuses of mammals (e.g. mice, rats, monkeys and humans) and is highly ape-dependent in numerous species. The fetal wound healing is most likely due to the immature immune system. Indeed, recent studies have demonstrated that the absence of inflammation in fetal wounds leads to the efficient and scarless repair (Szpaderska, A. M., et al., Surgery. 137(5) (2005) 571-3). Therefore, the acceleration of skin repair taken together with anti-inflammatory functions (FIG. 2A-C) suggest that BHA may be able to attenuate the scar formation at the injured sites.

In this study, a range of acylated LMW-HA derivatives (AHA, DHA and BHA) were developed for excisional wound healing. BHA significantly accelerated skin repair, which could not be achieved by either AHA or DHA, indicating that the longer acyl chain of the N-butyryl group plays an important role in LMW-HA-mediated healing effects. In addition, when compared to a commercial wound care product (containing 5 mg/mL carboxymethyl chitosan), BHA was able to significantly promote dermal healing at a lower dose (0.25 mg/mL). This therapeutic effect was mainly due to the fact that BHA can modulate pro-inflammation, promote epithelialization and neovascularization, and remodel collagens. These results demonstrate that BHA promotes healing of both acute and chronic wounds. In one embodiment, a composition that includes a LMW-HA derivative (e.g., BHA-gel) is applied to a wound alone or in combination with commercial wound care products to promote healing efficiency.

In other embodiments of the disclosure, the hyaluronic acid derivatives are formulated as pharmaceutical compositions comprising a hyaluronic acid derivative as described herein and a pharmaceutically acceptable excipient or carrier. In various embodiments, the pharmaceutical composition comprises one or more compounds of the present disclosure and one or more, pharmaceutically acceptable carriers and/or diluents and/or adjuvants and/or excipients, and optionally a therapeutic agent.

The hyaluronic acid derivatives described herein are compatible with mixing in a suitable vehicle in which the derivative is either dissolved or suspended. The derivatives may be dissolved in water, salt solutions, other pharmaceutically acceptable solvents, either alone or in combination with compatible nutrients, antibiotics, or combined with other medications.

In one embodiment, the hyaluronic acid derivatives of the disclosure can be admixed with a preparation of the parent hyaluronan (hyaluronic acid) in a solution or suspension and will be compatible with mixing in a suitable vehicle in which the active ingredient is either dissolved or suspended. The parent hyaluronan may be derived from a mammalian or bacterial source or may be synthetic.

The hyaluronic acid derivatives of the disclosure can be administered to an animal in an effective, therapeutic amount, by various routes of administration, including but not limited to: orally, topically, subcutaneously, intramuscularly, intravenously, trans-dermally, intra-articularly, rectally, by colonic enema, in a mouth wash, in a gingival ointment, or by bladder instillation. The hyaluronic acid derivatives of the disclosure may be mixed with food or feed or may be administered in a suitable vehicle, in which the active ingredient is either dissolved or suspended. Solution compositions may be water, salt solutions, and other solvents either alone or in combination with compatible nutrients, antibiotics, drugs suited to the condition, including the medical condition of the mammal.

A hyaluronic acid derivative of the present disclosure may be orally administered, for example, with an inert diluent or with an assimilable edible carrier, or it may be enclosed in hard or soft, shell gelatin capsules, or it may be compressed into tablets, or it may be incorporated directly with the food of the diet. For oral therapeutic administration, the derivative may be incorporated with excipient and used in the form of ingestible tablets, buccal tablets, troches, capsules, elixirs, suspensions, syrups, wafers, and the like. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet.

A hyaluronic acid derivative of the present disclosure may also be administered parenterally. Solutions of a derivative of the present disclosure can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations.

The hyaluronic acid derivatives of the disclosure may be admixed with naturally occurring hyaluronan (hyaluronic acid) of any molecular weight and viscosity, of mammalian or bacterial origin, for the purpose of administering to a mammal by any of the said routes above as a method of treatment for a chronic or acute inflammatory condition or a degenerative condition in a mammal.

Kits and commercial packages for use in the therapeutic, diagnostic and research applications described herein are also within the scope of the present disclosure. In one embodiment, a kit or commercial package may comprise a hyaluronic acid derivative of the present disclosure or a composition comprising a derivative of the present disclosure together with instructions for using the kit. Further, the kit may comprise one or more reagents, buffers, packaging materials, and containers for holding the components of the kit.

In embodiments of the disclosure, the hyaluronic acid derivatives are prepared from hyaluronic acid by removal of the N-acetyl group, —C(O)CH₃, resulting in deacetylated hyaluronic acid having a free amino group, —NH₂, as a derivative of the disclosure. In one embodiment, the acetyl group is removed using a hydrazinolysis reaction, by reacting, for example, hyaluronic acid with hydrazine or a hydrazine containing reactant.

Other acyl-substituted derivatives of the present disclosure can be prepared from the deacetylated hyaluronic acid by addition of an acyl group, for example, by using an acyl-donating reactant. Examples of acyl-donating reactants include acyl-anhydride or acyl chlorides, such as for example, butyryl anhydride, butyryl chloride etc. Acyl substituted hyaluronic acid derivatives of the present disclosure are isolated from the reaction of the deacetylated hyaluronic acid and the acyl-donating group.

In certain embodiments, the portion of N-acetyl groups which are removed and subsequently replaced with other acyl groups or hydrogen is dependent on the amount of hydrazine (or a hydrazine containing reactant) which is used in the deacetylation reaction and the length of time the reaction is allowed to proceed. Higher concentrations of hydrazine (or a hydrazine containing reactant) and/or longer reaction times, will increase the portion of acetyl groups which are removed from the hyaluronic acid, and subsequently increase the yield of the hyaluronic acid derivatives of the present disclosure. In further embodiments, the portion of acetyl groups which are replaced with other acyl groups or hydrogen (or reacetylated) is also dependent upon the amount of acyl donating group which is used in the reaction to form the acyl-substituted derivatives, as well as the length of time the reaction is allowed to proceed. Higher concentrations of the acyl donating reactant and/or longer reaction times, will increase the portion of acyl-substituted groups in the hyaluronic acid derivatives.

In another embodiment, cross-linked hyaluronic acid derivatives are prepared by adding a cross-linker to the hyaluronic acid derivatives of the present disclosure. In one embodiment, the degree of gelation of the cross-linked hyaluronic acid derivatives (or polymers) is controlled by adjusting the concentrations of the hyaluronic acid derivatives and/or the cross-linker, or by selection of the cross-linker. in one embodiment, the cross-linker is genipin.

In another embodiment, the cross-linking is achieved by polymerizing the cross-linking agent, such as genipin, in the presence of the hyaluronic acid derivatives, such that the cross-linking agent is an oligomer or macromer. In another embodiment, the cross-linking reaction is conducted in the presence of connective tissue components such as collagens, other matrix proteins and glycoproteins.

The hyaluronic acid derivatives of the present disclosure are useful for wound healing including reduction of inflammation, and conditions in which the modulation or inhibition of the production of inflammatory cytokines is beneficial. A person skilled in the art would understand that increased cytokine production plays an important role in conditions in which inflammation plays a role. The derivatives of the present disclosure are able to modulate or inhibit the production of inflammatory cytokines, which occurs as a result of stimulation by, for example, lower molecular mass hyaluronan, lipopolysaccharide or other microbial stimulants.

In macrophage cell culture tests, the hyaluronic acid derivatives of the present disclosure have been found to be non-toxic to the cells, and generally do not elicit an immune response.

In one embodiment of the disclosure there is included a method for the treatment of wound healing comprising administering to a patient in need thereof a hyaluronic acid derivative or composition as described herein, in another embodiment, there is included a use of a hyaluronic acid derivative or composition as described herein for the treatment of inflammation. in one embodiment, the inflammation results from the production of pro-inflammatory cytokines in the patient in one embodiment, the pro-inflammatory cytokines are selected from IL1-β, IL6, IL8, MCP1, and TNFα. Such conditions include, for example, repair of tissue and repair to loss of substance of the skin for medical or cosmetic reasons. In one embodiment, the cross-linked hyaluronic derivatives may be also fashioned in sheets of required size and width for the purposes of covering burns and grafts in plastic and cosmetic surgery.

In another embodiment, the disclosure includes a method for the treatment of a disease associated with the release of cytokines comprising administering to a patient in need thereof a hyaluronic add derivative or composition as described herein, wherein the cytokines have the potential to cause damage to organs in a mammal. In one embodiment, the disease associated with the release of cytokines is a bacterial infection, such as septic shock.

In another embodiment of the disclosure, the cross-linked hyaluronic acid derivatives are injected, or introduced by surgical procedures, including arthroscopic, endoscopic procedures and computer guided imaging, into soft tissue or hard tissue, such as cartilage or bone. Such surgical procedures include those used in cosmetic and reconstructive surgery.

In another embodiment, the cross-linked hyaluronic add derivatives (gels) are prepared as sheets to be used in applications where large surface areas are covered. For example, the gels are prepared as sheets having a desirable size and consistency, wherein the sheets are used to treat burns or are used in skin graft operations. In another embodiment, the cross-linked hyaluronic add derivatives are used as artificial matrices to grow cells, such as skin fibroblasts or other skin cells, so as to produce artificial skin to be used in burns or skin graft operations.

In another embodiment, the cross-linked hyaluronic acid derivatives can be cross-linked or other-wise incorporated into artificial polymers suitable for optical lenses and utilized in the production of said optical lenses.

### EXAMPLES

The following working examples further illustrate the invention and are not intended to be limiting in any respect.

### Materials and Methods

BHA was synthesized and characterized as described previously (Babasola, O., et at., J. Biol. Chem. 289(36) (2014) 24779-91). BHA solutions were prepared in sterilized ultra-pure water, and the endotoxin level in BHA solutions was measured using an Endotoxin Assay kit (GenScript, USA) according to the manufacturer's instructions. In addition, a gel formulation was prepared for in vivo studies. Briefly, 30 g of alginate, 450 g of glycerin. 2 g of ethylparaben, and 0.5 g of calcium gluconate were prepared in 1 L of sterilized ultra-pure water to produce the Blank-Gel. In addition, BHA solutions prepared as described above were added into the Blank-Gel to form the BHA-Gel. The animal ethics committee of Jilin University approved all experiments. Male Wistar rats (˜200 g, purchased from Changchun Institute of Biological Products, China) were maintained in a pathogen free animal facility for 2 weeks prior to the experiments.

### Example 1

### Therapeutic Efficacy of BHA

Four excisional full-thickness wounds (˜1.78 cm²) were made deep into the dermis of each Male Wistar rat without damaging the subdermal vasculature on the dorsal surface with disinfected surgical scissors. Six hours after surgery (Day 0), animals were randomly divided into 4 groups: untreated control group, Blank-Gel (negative control group), carboxymethyl chitosan (CMC) ([c] of CMC=5 mg/mL in CHITIN®, a commercial wound care product purchased from Shijiazhuang Yishengtang Medical Supplies Ltd., China) (positive control group), and BHA-Gel ([c] of BHA=0.05, 0.1, 0.25, 0.5 and 1 mg/mL). Animals were treated daily with 0.2 mL of Blank-Gel, CMC and BHA-Gel (see Table 1), and the wound diameter was measured at Day 3, 5, 7, 10 and 14. The healing rate was calculated as (1-Sn/S₀)×100%, where Sn=the wound surface area at a predetermined day, S₀=the wound surface area at Day 0.

### Example 2

### Therapeutic Mechanisms of BHA

### In Vitro Studies

The HUVEC (Human Umbilical Vein Endothelial Cells) cell line was purchased from the American Type Culture Collection (ATCC, USA). Ceils were maintained in RPMI-1640 medium (Corning) containing 10% fetal bovine serum (FBS; Gibco) and a Penicillin-Streptomycin Nystatin solution (Biological Industries) at 37° C. under 5% CO₂ atmosphere.

Cell proliferation was examined using Matrigel-based (a liquid laminin/collagen gel) Endothelial Cell Tube Formation Assay (Skovseth D. K. et al., Methods. Mol. Biol. 360 (2007) 253-68). Briefly, 200 μL of Matrigel (Corning) per well were added into 24-well plates. When the Matrigel solidified, HUVEC were seeded at a density of 3x 10 cells per well for 24 h. Cells were then treated with 0.05 mg/mL BHA in fresh growth medium for 48 h. Subsequently, cells were observed using a microscope (Olympus BX53).

Cell migration was studied using the scratch assay (Jonkman, J. E., et al., Cell Adh. Migr. 8 (5) (2014) 440-51). HUVEC were seeded in 6-well plates at a density of 4×10⁵ cells per well to reach 100% confluence. The scratches were made by pipette tips through the monolayer in the middle of the plate. Cells were then treated with serum-free medium containing BHA at 0.05 mg/mL, After 48 h, images were obtained using a microscope (Olympus BX53).

### In Vivo Studies

Animals with excisional full-thickness wounds were prepared as described in Example 1. Rats were sacrificed on the predetermined day and the damaged tissues along with the surrounding (˜2 mm) healthy tissues were collected for the following investigations.

Determination of mRNA expression via RT-PCR was performed. Tissue homogenates obtained using a tissue grinder (Scientz, Ningbo, Zhejiang, China) were centrifuged at 15,000 rpm for 10 min at 4° C. to remove the insoluble debris, and the supernatant was collected for reverse transcription polymerase chain reaction (RT-PCR). Total RNA was isolated using the TriZol Up reagent (TransGen Biotech, Beijing, China) following the manufacturer's instructions. First-strand cDNA was obtained from total RNA samples using the TransScript® All-in-One First-Strand cDNA Synthesis SuperMix kit (TransGen Biotech, Beijing, China). Quantitative real-time RT-PCR was carried out using the StepOnePlus™ Real-Time PCR System (Thermo Scientific). RT-PCR was performed under the following conditions: an initial denaturation step at 94° C. for 30 s, followed by 45 cycles of 5 s at 94° C., annealing for 30 s at 60° C. The quantitative level of each target mRNA was measured as a fluorescent signal corrected according to the signal for β-actin RNA.

Determination of protein expression via western blotting and ELISA was performed. Total protein was obtained using the RPA Lysis Buffer (GenStar, China) containing 1 mM PMSF (GenStar, China). Nuclear protein was obtained using the Nuclear and Cytoplasmic Protein Extraction Kit (Beyotime, China) containing 1 mM PMSF (Beyotime, China). Protein concentrations were determined using the BOA kit (TransGen Biotech, China): 20 μg of protein per sample were loaded onto an SDS-polyacrylamide gel and electrophoresed at 100 V for 2 h. Protein was then transferred to a polyvinylidene difluoride (PVDF) membrane (Millipore) for 1.5 h at 200 mA. Membranes were incubated overnight with appropriate primary antibodies [anti-TGF-β1 antibody (ab179695), anti-TAK-1 antibody (ab109526), anti-p-TAK-1 antibody (ab109404) and anti-p38 antibody (ab107799), Abcam, USA: Anti-p-p38 antibody (AF4001), anti-Collagen I antibody (AF7001), anti-Collagen III antibody (AF0136), anti-β-actin antibody (AF7018), Affinity, USA] at 4 C. Antibody reactive bands were detected with HRP-labeled secondary antibodies using EasySee® Western Blot kit (TransGen Biotech, China). In addition, the concentrations of TNF-α, IL-6 and IL-1β were determined using the Rat TNF-α ELISA kit, Rat interleukin 6 ELISA kit, and Rat interleukin 1β ELISA kit (Cusbio, China).

Histopathological examinations were performed. Skin biopsies were fixed in 4% paraformaldehyde (PFA), embedded in paraffin, and sectioned (4 μm). Sections were treated with hematoxylin-eosin (H&E) and Masson's trichrome stains, respectively. Inflammatory cell infiltration, fibroblast proliferation, blood vessel formation, hair follicle formation and collagen deposition were observed under a microscope (Olympus BX53). To quantify the inflammatory cell infiltration and collagen deposition, integrated optical density (IOD), the area of positive regions (A) and IOD/A of each slide (n=6) were analyzed using image-Pro Plus 6.0 software (Media Cybernetics, Inc., USA), and the mean was calculated based on IOD/A. To quantify the fibroblast proliferation, three epidermis areas of each slide (n=6) were selected randomly and analyzed using Image-Pro Plus 6.0 software, and the mean was calculated based on the epidermal thickness. In addition, the development of blood vessels and formation of hair follicles were quantified based on the mean of new blood vessels and hair follicles in slides (n=6).

In addition, dewaxed sections were immediately immersed in 3% H₂O₂ to block endogenous peroxidase, and the antigen retrieval was performed using 0.01 M citrate buffer, pH 6.0 (Maxim, Fuzhou, China). The sections were then blocked in 5% BSA (Roche). Primary antibodies including anti-CD31 antibody (ab182981, Abcam, USA), anti-LYVE-1 antibody (N8600-1008SS, Novus) and anti-CD44 antibody (ab189524, Abcam, USA) were incubated overnight at 4° C., followed by the incubation with HRP-labeled secondary antibodies (Affinity, USA). After counterstaining with hematoxylin, positively stained cells were observed under a microscope (Olympus BX53). To quantify the antigens, integrated optical density (IOD), the area of positive regions (A) and IOD/A of each slide (n=6) were analyzed using image-Pro Plus 6.0 software, and the mean was calculated based on IOD/A.

### Example 3

### Statistical Analysis

Data was calculated as the mean±standard deviation (SD). An unpaired Student's t-test (two-tailed) was used to test the significance of differences between two mean values. A one-way ANOVA (Bonferroni's Post-Hoc test) was used to test the significance of differences in three or more groups. In all experiments, p<0.05 was considered statistically significant.

### Equivalents

It will be understood by those skilled in the art that this description is made with reference to certain embodiments and that it is possible to make other embodiments employing the principles of the invention which fall within its spirit and scope.

1. A composition for promoting wound healing in tissue of a subject, comprising:
a pharmaceutically acceptable excipient or carrier, and
a therapeutically effective amount of a hyaluronic acid derivative comprising repeating units of a disaccharide of Formula (I), wherein a portion of the disaccharide units of Formula(I) have been independently replaced with a disaccharide structure of Formula (II) wherein R is —C(O)—(C₂-C₂₀)-alkyl, —C(O)—(C₂-C₂₀)-alkenyl or —C(O)—(C₂-C₂₀)-alkynyl, or a pharmaceutically acceptable sodium- or potassium-salt, ester, or glucoside thereof, 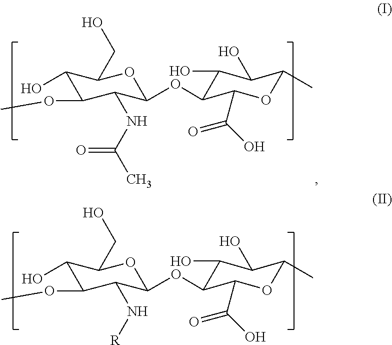
wherein the hyaluronic acid derivative has a molecular weight of at least about 20 kDa, and
wherein the hyaluronic acid derivative promotes wound healing.

2. (canceled)

3. The composition of claim 1, wherein the hyaluronic acid derivative is cross-linked.

4. The composition of claim 1, wherein R is —C(O)—(C₂₋₄)-alkyl.

5. The composition of claim 1, wherein the portion of N-acetyl groups which are replaced is at least about 10%.

6. The composition of claim 1, wherein the portion of N-acetyl groups which are replaced is between about 20% to about 80%.

7. The composition of claim 1, wherein the molecular weight is at least about 30 kDa.

8. The composition of claim 1, wherein the molecular weight is between about 20 kDa to about 250 kDa.

9. The composition of claim 1, wherein the amount of hyaluronic acid derivative is about 0.05 to about 1 mg/mL.

10. The composition of claim 1, wherein the amount of hyaluronic acid derivative is 0.25 mg/mL.

11. The composition of claim 1, further comprising moisturizer, emollient, thickener, preservative, and/or a firming agent.

12. The composition of claim 11, wherein the moisturizer is glycerol, petroleum jelly, petrolatum, propylene glycol, butylene glycol, Lactic acid or a salt thereof, erythriol, D-panthenol, PEG-n, or 2-methacryloyloxyethyl phosphorylcholine.

13.-14. (canceled)

15. The composition of claim 11, further comprising sodium carboxymethylcellulose, or 1-(3-dimethylaminopropyl)-3-ethyl-carbodiimide hydrochloride.

16. The composition of claim 11, wherein the thickener is sodium alginate, an anionic polysaccharide, alginic acid, alginates, pectin, carrageenan, xanthan gum, carboxy methyl cellulose, a cationic polysaccharide, chitosan, polyquanternium-4, polyquanternium-10, a non-ionic polysaccharide, guar gum, hydroxypropyl guar, locust bean gum, sclerotium, methyl cellulose, hydroxyethyl cellulose, carbopol, acrylate copolymer, mineral salt, magnesium aluminium silicate, and/or a surface active agent, potassium stearate, betaine.

17. The composition of claim 11, wherein the preservative is ethyl hydroxybenzoate, hydroxyphenyl or an ester or salt thereof, methylparaben, benzylparaben, sodium methylparaben, sodium butylparaben, isothiazolinone, methyltchloroisothiazolinone, methylisothiazolinone, acidic preservative, benzoic acid, sorbic acid, alcohol, bronopol, phenoxyethanol, quaternary ammonium salt, benzalkonium chloride, benzethonium chloride, aldehyde, (benzyloxy)methanol, glutaric dialdehyde, phenol, chlorophene, chloroxylenol, or a combination thereof

18. The composition of claim 11, wherein the firming agent is calcium gluconate, calcium chloride.

19. The composition of claim 11, wherein the firming agent is calcium gluconate, calcium chloride.

20. The composition of claim 1, wherein the wound comprises a diabetic wound, canker, ulcer, aphthous stomachitis, sore, burn, surgical wound, bite, abrasion, surgical adhesion, and/or tissue damage due to chemical damage, thermal damage, or trauma.

21. The composition of claim 1, wherein the wound is a chronic wound.

22. The composition of claim 1, wherein the wound is an acute wound.

23. A method for promoting wound healing in a subject, the method comprising: administering to the subject a formulation that comprises a hyaluronic acid comprising repeating units of a disaccharide comprising glucuronic acid and N-acetylglucosamine, wherein a portion of the N-acetyl groups of the N-acetylglucosamine have been independently replaced with a group of the formula formula —N—C(O)—(C₂-C₂₀)-alkyl, —N—C(O)—(C₂-C₂₀)-alkenyl or —N—C(O)—(C₂-C₂₀)-alkynyl, and wherein the hyaluronic acid derivative has a molecular weight of at least about 20 kDa, or a pharmaceutically acceptable sodium or potassium salt, ester, or glucoside thereof.

24.-29. (canceled)
